(19) United States
(12) Patent Application Publication
(10) Pub. No.: US 2015/0047077 A1
(43) Pub. Date: Feb. 12, 2015

(54) GLORY 583 LETTUCE VARIETY

(71) Applicant: PROGENY ADVANCED GENETICS, Salinas, CA (US)

(72) Inventor: Darryn GIBSON, Prunedale, CA (US)

(21) Appl. No.: 14/453,591

(22) Filed: Aug. 6, 2014

Related U.S. Application Data
(60) Provisional application No. 61/862,876, filed on Aug. 6, 2013.

(51) Int. Cl. A01H 5/12

(52) U.S. Cl. CPC A01H 5/12
USPC 800/305; 435/410

(57) ABSTRACT

A new lettuce variety designated ‘Glory 583’ is described. ‘Glory 583’ is an iceberg lettuce variety exhibiting stability and uniformity.

### CROSS REFERENCE TO RELATED APPLICATION

This application claims the benefit under 35 § USC 119(e) of prior co-pending U.S. Provisional Patent Application No. 61/862,876, filed Aug. 6, 2013, the disclosure of which is hereby incorporated by reference in its entirety.

### FIELD

This invention relates to the field of plant breeding. In particular, this invention relates to new lettuce, Lactuca sativa, variety, ‘Glory 583’.

### BACKGROUND

Lettuce is an increasingly popular crop. Worldwide lettuce consumption continues to increase. As a result of this demand, there is a continued need for new lettuce varieties. In particular, there is a need for improved romaine lettuce varieties that exhibit improved growth habits, bolting and tip burn tolerance, and disease resistance.

### SUMMARY

In order to meet these needs, the present invention is directed to an improved iceberg lettuce variety that exhibits vigorous growth, increased weight, and increased yield. In particular, the present invention is directed to lettuce, Lactuca sativa, seed designated as ‘Glory 583’ having ATCC Accession Number X1. The present invention is further directed to a lettuce head isolated from a Lactuca sativa plant produced by growing ‘Glory 583’ lettuce seed having ATCC Accession Number X1. The present invention is further directed to a Lactuca sativa plant having all the physiological and morphological characteristics of a Lactuca sativa plant produced by growing ‘Glory 583’ lettuce seed having ATCC Accession Number X1. The present invention is further directed to an F₁ hybrid Lactuca sativa plant having ‘Glory 583’ as a parent, where ‘Glory 583’ lettuce seed is grown from ‘Glory 583’ seed having ATCC Accession Number X1.

The present invention is further directed to lettuce, Lactuca sativa plants and lettuce heads isolated therefrom produced by growing ‘Glory 583’ lettuce seed. The present invention is further directed to a Lactuca sativa plant and the lettuce head isolated therefrom having all the physiological and morphological characteristics of a Lactuca sativa plant produced by growing ‘Glory 583’ lettuce seed having ATCC Accession Number X1. The present invention is further directed to an F₁ hybrid lettuce, Lactuca sativa plant and a head isolated therefrom grown from the seed having ‘Glory 583’ as a parent wherein ‘Glory 583’ is grown from ‘Glory 583’ lettuce seed having ATCC Accession Number X1.

The present invention is further directed to pollen isolated from ‘Glory 583’ lettuce plants. The present invention is further directed to ovules isolated from ‘Glory 583’ lettuce plants. The present invention is further directed to tissue culture of ‘Glory 583’ lettuce plants.

The present invention is further directed to a method of selecting lettuce plants by: a) growing more than one ‘Glory 583’ lettuce plant, where the plants are grown from lettuce seed having ATCC Accession Number X1; and b) selecting a plant from step a). The present invention is further directed to lettuce plants and seeds produced therefrom, where the lettuce plants and seeds are isolated by the selection method of the invention.

The present invention is further directed to a method of breeding lettuce by crossing a lettuce plant with a plant grown from ‘Glory 583’ lettuce seed having ATCC Accession Number X1. The present invention is further directed to lettuce plants, heads isolated therefrom, and seeds produced therefrom, where the lettuce plant is isolated by the breeding method of the invention.

### DETAILED DESCRIPTION

### Definitions

In order to more clearly understand the invention, the following definitions are provided:

Iceberg Lettuce: Iceberg lettuce, Lactuca sativa L. var. capitala L. is also known as ‘crisp head’ lettuce. Iceberg lettuce is a lettuce plant type that forms a firm, spherical head formed with tightly folded brittle textured foliage. Internal color ranges from white to yellow to light green. The wrapper leaves surrounding the head are wider than they are long. Leaf margins can vary by type, being entire, undulating, or frilled. Wrapper leaf color ranges from yellow green to dark green.

Core Length: Core length is the length of the internal lettuce stem. Core length is measured from the base of the cut head to the tip of the core.

Core Diameter: Core diameter is the diameter of the lettuce stem at the base of the cut head.

Head Diameter: Head diameter is the diameter of the vertically sliced lettuce plant head at its widest horizontal point, perpendicular to the stem.

Head Length: Head length is the diameter of the vertically sliced lettuce plant head as measured from the base of the cut stem to the cap leaf.

Average Head Diameter: Average head diameter is an average of the measured head diameter and head length of the lettuce head.

Average Head Diameter: Core Length Ratio: The ratio of the average head diameter to core length is indicative of the percentage of useable product produced by the lettuce plant.

Frame Diameter: The frame diameter is a measurement of the lettuce plant diameter at its widest point. The measurement of frame diameter is from the outer most wrapper leaf tip to outer most wrapper leaf tip.

Head Weight: Head weight is the weight of the marketable lettuce plant, cut and trimmed to market specifications.

Rogueing: Rogueing is the process in lettuce seed production where undesired plants are removed from a variety. The plants are removed because they differ physically from the general desired expressed characteristics of the variety. The differences can be related to size, color, maturity, leaf texture, leaf margins, growth habit, or any other characteristic that distinguishes the plant.

Market Stage: Market stage is the stage when a lettuce plant is ready for commercial lettuce harvest. In the case of a romaine lettuce variety, a romaine plant is at a marketable state when the heart has some density and the head has reached an adequate size and weight.

Taking into account these definitions, the present invention is directed to seeds of the lettuce variety ‘Glory 583’, plants produced by growing ‘Glory 583’ lettuce seeds, head isolated or harvested from the plants, one or more plants selected from a collection of ‘Glory 583’ plants and seeds derived or produced therefrom; plants produced by crossing a lettuce plant with a ‘Glory 583’ lettuce plant and seeds derived or produced therefrom.

‘Glory 583’ is an iceberg lettuce variety that is distinct to all known iceberg lettuce varieties due to its large heading, and improved heat resistance for plantings in regions such as Yuma, Ariz. and California's San Joaquin and Imperial valleys.

Through extensive field trialing and screenings, ‘Glory 583’ has demonstrated to have a large heading, and to be well adapted for fall plantings in regions such as Yuma, Ariz. and California's San Joaquin and Imperial valleys.

‘Glory 583’ was evaluated in multiple trials and evaluated for improved size, improved weight, improved frame size, and improved tolerances to tip burn and core length.

‘Glory 583’ has been extensively trialed in Yuma, Ariz., Huron, Calif., and the San Joaquin valley of California, and was selected for improved size, tolerances to tip burn and slower maturity. ‘Glory 583’ was also selected for growth during fall plantings in Yuma, Ariz., Huron, Calif., and the San Joaquin valley of California.

As evaluated in multiple seed production fields and commercial plantings, ‘Glory 583’ has been observed to be uniform and stable without variants.

As described herein, lettuce variety ‘Glory 583’ has numerous distinguishing characteristics.

A. Variety Description Information

| | Plant Type: |
|---|---|
| | Iceberg |
| Seed: | |
| Seed Color: | Black |
| Light Dormancy: | No |
| Heat Dormancy: | Yes |
| Cotyledons: | |
| Shape of Cotyledons: | Intermediate |
| Shape of Fourth Leaf: | Spatulate |
| Length/Width Index of Fourth Leaf: | 20 |
| Apical Margin: | Moderately Dentate |
| Basal Margin: | Finely Dentate |
| Undulation: | Slight |
| Green Color: | Dark Green |
| Anthocyanin: | |
| Distribution: | None |
| Rolling: | Absent |
| Cupping: | Uncupped |
| Reflexing: | Slight |
| Mature Leaves: | |
| Margin: | |
| Incision Depth (Deepest penetration of | Deep |
| the margin): | |
| Indentation (Finest Division of the | Crenate |
| Margin): | |
| Undulation of the Apical Margin: | Moderate |
| Green Color: | Dark |
| Anthocyanin | Absent |
| Distribution: | None |
| Size: | Large |
| Glossiness: | Moderate |
| Blistering: | Moderate |
| Leaf Thickness: | Thick |
| Trichomes: | Absent |

B. Comparison to Most Similar Variety

| Characteristic | Glory 583 | Sun Devil |
|---|---|---|
| Spread of Frame Leaves | 47 cm | 48 cm |
| Head Diameter (market trimmed with | 15 cm | 13 cm |
| single cup leaf) | | |
| Head Shape | Spherical | Spherical |
| Characteristic | Glory 583 | Sun Devil |
| Head Size Class | large | Large |
| Head Count per Carton | 24 | 24 |
| Head Weight | 1006 | 848 |
| Head Firmness | Firm | Firm |
| Butt | | |
| Shape | Rounded | Rounded |
| Midrib | Moderately | Moderately |
| | Raised | Raised |
| Core (Stem of Market-trimmed Head) | | |
| Diameter at the base of the Head | 4.0 cm | 4.5 cm |
| Ratio of Head Diameter/Core Diameter | 3.75 | 2.9 |
| Core Height from base of Head to Apex | 3.0 cm | 3.5 cm |
| Number of Days from First Water Date | 61 | 60 |
| to Seed Stalk Emergence (Summer | | |
| condition) | | |
| Bolting Class | Slow | Slow |
| Height of Mature Seed Stalk | 105 cm | 119 cm |
| Spread of Bolter Plant | 35 cm | 41 cm |
| Bolter Leaves | Curved | Curved |
| Margin | Dentate | Dentate |
| Color | Dark Green | Medium Green |
| Bolter Habit | | |
| Terminal Inflorescence | Present | Present |
| Lateral Shoots (above head) | Absent | Present |
| Basal Side Shoots | Absent | Present |
| Adaptation Regions | Huron, CA. | Huron, CA. |
| | Yuma, AZ. | Yuma, AZ. |

C. Growing Season

| Season | ‘Glory 583’ | ‘Sun Devil’ |
|---|---|---|
| Spring area | Not Adapted | Not Adapted |
| Summer area | Not Adapted | Not Adapted |
| Fall area | Desert Southwest | Desert Southwest |
| Winter area: | Not Adapted | Not Adapted |

D. Diseases and Stress Reactions

| Disease or Stress | ‘Glory 583’ | ‘SunDevil’ |
|---|---|---|
| Virus | N/A | N/A |
| Big Vein: | Susceptible | Susceptible |
| Lettuce Mosaic: | Resistant | Resistant |

E. Fungi/Bacteria

| Fungal/Bacterial | ‘Glory 583’ | ‘Sun Devil’ |
|---|---|---|
| Corky Root Rot (Pythium | Susceptible | Susceptible |
| Root Rot): | | |
| Downy Mildew (Races I, | Susceptible | Susceptible |
| IIA, III): | | |
| Powdery Mildew: | Susceptible | Susceptible |
| Sclerotinia Rot: | Susceptible | Susceptible |
| Bacterial Soft Rot | Susceptible | Susceptible |
| (Pseudomonas spp. & | | |
| others): Not tested | | |
| Botrytis (Gray Mold): | Susceptible | Susceptible |
| Other: Corky Root Rot | Susceptible | Susceptible |
| (Rhizomonas suberifaciens): | | |

F. Insects

| Insects | ‘Glory 583’ | ‘Sun Devil’ |
|---|---|---|
| Cabbage Loopers: | Susceptible | Susceptible |
| Root Aphids: | Susceptible | Susceptible |
| Green Peach Aphid: | Susceptible | Susceptible |

G. Physiological/Stress

| Stress | ‘Glory 583’ | ‘Sun Devil’ |
|---|---|---|
| Tipburn | Resistant | Resistant |
| Heat | Resistant | Resistant |
| Drought | Susceptible | Susceptible |
| Cold | Susceptible | Susceptible |
| Salt | Susceptible | Susceptible |

H. Post Harvest

| Characteristic | ‘Glory 583’ | ‘Sun Devil’ |
|---|---|---|
| Pink Rib | Moderately Susceptible | Moderately Susceptible |
| Russet Spotting | Moderately Susceptible | Moderately Susceptible |
| Rusty Brown | Moderately Susceptible | Moderately Susceptible |
| Discoloration | | |
| Internal Rib Necrosis | Moderately Susceptible | Moderately Susceptible |
| (Blackheart, Gray | | |
| Rib, Gray Streak) | | |
| Brown Stain | Moderately Susceptible | Moderately Susceptible |

### Breeding and Selection

The present invention is further directed to the use of ‘Glory 583’ lettuce in breeding and selection of new varieties.

A. Breeding

In lettuce breeding, lines are selected for their appropriate characteristics. For example, one line may be selected for bolt tolerance in the fall growing conditions of the desert production locations of California and Arizona. Another line may be selected for the size, color, and texture of the lettuce head. Crosses are made, for example, to produce a dark green, sure heading iceberg lettuce with improved texture, and size for fall plantings in Yuma, Ariz. and Huron, Calif.

To optimize crossing, it is important to note that lettuce is an obligate self-pollinating species. This means that the pollen is shed before stigma emergence, assuring 100% self-fertilization. Since each lettuce flower is an aggregate of about 10-20 individual florets (typical of the Compositae family), manual removal of the anther tubes containing the pollen is performed by procedures well known in the art of lettuce breeding.

The manual removal of anther tubes, though an effective means to ensure the removal of all self pollinating possibilities, is very tedious and time consuming when a large number of crosses are to be made. The breeders have therefore adapted a well documented and modified method of making crosses more efficiently using these methods. This particular cross was made by first misting the designated male flowers to wash the pollen off prior to fertilization. This process of misting is a proven and effective means of pollen removal that assures crossing or hybridization. About 60-90 minutes past sunrise, flowers to be used for crossings are selected. The basis for selection are open flowers, with the stigma emerged and the pollen visibly attached to the single stigma (about 10-20 stigma). Using 3-4 pumps of water from a regular spray bottle, the pollen is washed off with enough pressure to dislodge the pollen grains, but not enough to damage the style. Excess water is dried off with clean paper towels. About 30 minutes later, the styles should spring back up and the two lobes of the stigma are visibly open in a “V” shape. Pollen from another variety or donor parent is then introduced by gently rubbing the stigma and style of the donor parent to the maternal parent. Tags with the pertinent information on date and pedigree are then secured to the flowers in order to keep track.

About 2-3 weeks after pollination, seeds are harvested when the involucre have matured. The seeds are eventually sown and in the presence of markers such as leaf color or leaf margins, the selfed or maternal seedlings or plants are identified. Generally, there are no visible markers and breeders must wait until the F₂ generations when expected segregation patterns for the genetic character of interest can be followed. This latter situation mandates a lengthy wait to determine if hybrids are produced. Two relevant references teaching methods for out crossing lettuce are: (1) Ryder, E. J. and A. S. Johnson. 1974. Mist depollination of lettuce flowers. Hortscience 9:584; and (2) Nagata, R. T. 1992. Clip and Wash Method of Emasculation for Lettuce. Hortscience 27(8):907-908 both of which are hereby incorporated by reference in their entirety for the purpose of providing details on the techniques well known in the art.

B. Selection

In addition to crossing, selection may be used to identify and isolate new lettuce lines. In lettuce selection, lettuce seeds are planted, the plants are grown and single plant selections are made of plants with desired characteristics. Such characteristics may include improved head and frame size, deeper or darker green leaf color, etc. Seed from the single plant selections are harvested, separated from seeds of the other plants in the field and re-planted. The plants from the selected seed are monitored to determine if they exhibit the desired characteristics of the originally selected line. Selection work is continued over multiple generations to increase the uniformity of the new line.

### DEPOSIT INFORMATION

A deposit of the lettuce variety ‘Glory 583’ is maintained by Progeny Advanced Genetics, having an address at 590A Works Street, Salinas, Calif. 93901, United States of America. Access to this deposit will be available during the pendency of this application to persons determined by the Commissioner of Patents and Trademarks to be entitled thereto under 37 C.F.R. §1.14 and 35 U.S.C. §122. Upon allowance of any claims in this application, all restrictions on the availability to the public of the variety will be irrevocably removed by affording access to a deposit of at least 2,500 seeds of the same variety made according to the Budapest Treaty in the American Type Culture Collection, (ATCC), ATCC Patent Depository, 10801 University Boulevard, Manassas, Va., 20110, USA.

The lettuce variety ‘Glory 583’ was deposited on (DATE) according to the Budapest Treaty in the American Type Culture Collection (ATCC), ATCC Patent Depository, 10801 University Boulevard, Manassas, Va., 20110, USA. The deposit has been assigned ATCC number X1. Access to this deposit will be available during the pendency of this application to persons determined by the Commissioner of Patents and Trademarks to be entitled thereto under 37 C.F.R. §1.14 and 35 U.S.C. §122. Upon allowance of any claims in this application, all restrictions on the availability to the public of the variety will be irrevocably removed.

The deposit will be maintained in the ATCC depository, which is a public depository, for a period of at least 30 years, or at least 5 years after the most recent request for a sample of the deposit, or for the effective life of the patent, whichever is longer, and will be replaced if a deposit becomes nonviable during that period.

This invention will be better understood by reference to the following non-limiting Examples.

### EXAMPLES

### Example 1

### General Trialing Method

The following steps illustrate the general trialing method of the invention:

I. Set Up 1. A trial is set up to compare one or more lines. Parental lines and related varieties are identified.

2. Primary slots are identified.

3. Necessary accession lines are located and purchased/received from seed dealers or growers.

4. All varieties are assigned a number to maintain integrity and anonymity.

5. Trials are set up in with all necessary varieties. Variety arrangement for trial is diagramed.

II. Planting 1. Commercial plantings are located by contacting commercial growers during the planting slot recommended for the variety.

2. A field is located during commercial planting and the necessary rows and area is marked off.

3. Varieties are planted according to a diagram, generally in 100 foot ranges.

4. All varieties are planted in same manner to mimic the planting of the commercial variety as closely as possible.

5. A trial map is drawn diagramming the trial, the trial location in the field and directions to the field.

III. Maintenance 1. All varieties are treated identically. Plants are watered, fertilized, and treated to control pests in the same manner as other lettuce plants in the commercial field.

2. The trial is thinned to separate the plants for optimum growth.

### Example 2

### Comparative Analysis

‘Glory 583’ is a new and distinct variety of iceberg lettuce that most closely resembles the commercial variety ‘Sun Devil’ and the previously released but no longer available variety ‘Glory.’ ‘Glory 583’ is a vanguard type iceberg lettuce variety adapted to the fall harvest of the desert South West and Huron lettuce production regions of California and Arizona. ‘Glory 583’ is large heading and large framed, widely adapted variety, with improved heading characteristics, and improved resistance to tip burn and bolting.

‘Glory 583’ is earlier maturing, heavier heading and has a shorter core than ‘Sun Devil.’ Similar to ‘Sun Devil’, ‘Glory 583’ is adapted to the fall harvest in the desert South West production region, and has heavy leaf texture, slow bolting and excellent tip burn resistance. Unlike ‘Sun Devil’ or Glory, ‘Glory 583’ can be planted up to 5 days earlier in the fall planting period, withstanding a higher number of heat units without affecting the quality of the heading.

Although the foregoing invention has been described in some detail by way of illustration and examples for purposes of clarity and understanding, it will be obvious that certain modifications and alternative embodiments of the invention are contemplated which do not depart from the spirit and scope of the invention as defined by the foregoing teachings and appended claims.

What is claimed:

1. Lactuca sativa seed designated as ‘Glory 583’ having ATCC Accession Number X1.

2. A Lactuca sativa plant produced by growing the seed of claim 1.

3. A lettuce head isolated from the plant of claim 2.

4. A Lactuca sativa plant having all the physiological and morphological characteristics of the Lactuca sativa plant of claim 2.

5. An F₁ hybrid Lactuca sativa plant having ‘Glory 583’ as a parent where ‘Glory 583’ is grown from the seed of claim 1.

6. Pollen of the plant of claim 2.

7. An ovule of the plant of claim 2.

8. Tissue culture of the plant of claim 2.

9. A method of selecting lettuce, comprising:
a) growing more than one plant from the seed of claim 1; and
b) selecting a plant from step a).

10. A Lactuca sativa plant selected by the method of claim 9.

11. Lactuca sativa seed produced from the Lactuca sativa plant of claim 10.
